(19) United States
(12) Patent Application Publication
(10) Pub. No.: US 2025/0127521 A1
(43) Pub. Date: Apr. 24, 2025

(54) Medical Auxiliary Device for Joint Bone Replacement Surgery

(71) Applicant: HSIEN TSUNG LU, Taipei (TW)

(72) Inventors: Hsien Tsung Lu, Taipei (TW); Michael Lu, Taipei (TW); Brian Lu, Taipei (TW); Rina Lu, Taipei (TW)

(21) Appl. No.: 18/914,289

(22) Filed: Oct. 14, 2024

(30) Foreign Application Priority Data
Oct. 19, 2023 (TW) 112140017

(51) Int. Cl. A61B 17/15

(52) U.S. Cl. CPC A61B 17/151; A61B 17/155; A61B 17/157

(57) ABSTRACT

A medical auxiliary device for joint bone replacement surgery that allows clinicians to precisely, quickly, and conveniently cut joint bones while providing stable fixation during the surgical procedure. a first auxiliary device that fits the femur to be cut, with a first fitting surface at the top that conforms to the shape of the femur. One side of the first auxiliary device is equipped with a first cutting groove and multiple locking holes. The first cutting groove allows an orthopedic saw to cut the femur, and the locking holes allow screws to pass through and fix the first auxiliary device to the femur. A second auxiliary device is fixed to the tibia to be cut, with a second fitting surface at the bottom that conforms to the shape of the tibia. One side of the second auxiliary device is equipped with a second cutting groove and multiple fixation holes.

### FIELD OF THE INVENTION

The present invention relates to the technical field of medical auxiliary devices, particularly to a medical auxiliary device for joint bone replacement surgery that guides an orthopedic saw to cut joint bones and provides stable fixation during the surgical process.

### BACKGROUND OF THE INVENTION

In the medical field, knee joint disorders are common issues caused by trauma, degeneration, or osteoporosis. Currently, the primary treatment for knee joint disorders is Total Knee Arthroplasty (TKA), a surgery that replaces damaged bones and cartilage with prostheses made from artificial biomaterials, aiming to reconstruct the knee joint. TKA is considered a highly successful surgery and can effectively treat knee joint disorders. However, before placing the prosthesis, osteotomy tools are typically required to remove the damaged femoral and tibial surfaces, and sometimes the patellar joint surface, to ensure proper installation of the prosthesis. Since the femoral and tibial surfaces cannot be restored once surgically trimmed and removed, accurately reshaping these surfaces is a critical aspect of knee replacement surgery to ensure the prosthesis fits well with the patient's native bones post-surgery.

Existing osteotomy tools, such as those disclosed in Taiwan Patent No. M294922 for “Improved Ruler Structure for Total Knee Arthroplasty” and Taiwan Patent No. M310697 for “Surgical Instrument for Total Knee Arthroplasty,” allow the removal of damaged femoral and tibial surfaces. However, these osteotomy tools are complex with many components, making them difficult to manufacture, assemble, and operate. This results in prolonged operation times and increased production costs. These inconveniences indicate the need for improvement.

In view of this, the objective of this invention is to overcome the aforementioned problems by developing a medical auxiliary device for joint bone replacement surgery. This device allows clinicians to precisely, quickly, and conveniently perform osteotomies and provides stable fixation during the surgery, addressing the shortcomings of conventional osteotomy tools.

### SUMMARY OF THE INVENTION

The objective of the present invention is to provide a medical auxiliary device for joint bone replacement surgery that enables clinicians to precisely, quickly, and conveniently perform osteotomies on joint bones while providing stable fixation during surgery.

To solve the above problems and achieve the objectives, the technical solution of the present invention is as follows: a medical auxiliary device for joint bone replacement surgery, applied to the femur and tibia. The device includes: a first auxiliary device, which has a vertical plate that conforms to the femur to be cut and a horizontal plate extending laterally from the bottom of the vertical plate, also conforming to the femur. The top of the vertical and horizontal plates features a first fitting surface matching the shape of the femur, and one side of the vertical plate is equipped with a first cutting groove and multiple locking holes. The first cutting groove allows an orthopedic saw to cut the femur, and the locking holes allow screws to pass through and fix the first auxiliary device to the femur; and a second auxiliary device corresponding to the first auxiliary device, fixed to the tibia. The bottom of the second auxiliary device has a second fitting surface matching the tibia's shape, and one side of the second auxiliary device is equipped with a second cutting groove and multiple fixation holes. The second cutting groove allows the orthopedic saw to cut the tibia, and the fixation holes allow screws to pass through and fix the second auxiliary device to the tibia.

More preferably, the first cutting groove consists of a first guide edge positioned at the top of the first cutting groove, a second guide edge positioned below the first guide edge, two slot walls connecting perpendicularly to the guide edges to limit the operating direction and range of the orthopedic saw, and a saw blade opening that penetrates the vertical plate between the guide edges and the slot walls.

More preferably, the second cutting groove consists of a third guide edge positioned at the top of the second cutting groove, a fourth guide edge positioned below the third guide edge, two slot walls connecting perpendicularly to the guide edges to limit the operating direction and range of the orthopedic saw, and a saw blade opening that penetrates the second auxiliary device between the guide edges and the slot walls.

More preferably, the horizontal plate includes a notch through which cruciate ligaments can pass.

More preferably, the vertical plate and horizontal plate are integrally formed.

More preferably, the medical auxiliary device is manufactured using 3D printing technology, comprising the following steps: Step 1: Obtain the patient's individual 3D digital images of the femur and tibia; Step 2: Superimpose the 3D digital images of the femur and tibia with the 3D digital images of the first and second auxiliary devices; Step 3: Subtract the overlapping 3D digital images to obtain the first and second auxiliary devices that match the patient's femur and tibia.

The functions and effects achieved by this invention include the following:

The application of the first and second auxiliary devices allows clinicians to perform osteotomies on joint bones more precisely, quickly, and conveniently during surgery, making the operation process easier and more controllable, providing more precise and safe surgical outcomes.

The use of the first and second fitting surfaces ensures that the first and second auxiliary devices are tightly fitted to the joint bones, allowing the clinician to precisely remove the damaged femoral and tibial surfaces, ensuring proper installation and stable support of the prosthesis post-surgery, restoring normal joint function and providing excellent surgical outcomes.

The design of the first and second cutting grooves, along with the locking and fixation holes, accurately guides the orthopedic saw and screws, providing stable fixation during surgery, ensuring precision and safety. This makes the surgery easier for clinicians while providing a controllable operating environment, improving surgical efficiency and success rates.

The medical auxiliary device is custom-made using 3D printing technology, ensuring a perfect fit with the patient's bone structure, resulting in better surgical outcomes.

The vertical and horizontal plates are integrally formed, increasing the strength and stability of the medical auxiliary device, reducing the number of connection points between components, and minimizing potential failure points, thereby enhancing the durability and reliability of the device. This simplifies the manufacturing process and reduces production costs.

### BRIEF DESCRIPTION OF THE DRAWINGS

FIG. 1 is a perspective schematic diagram of the present invention;

FIG. 2 is a perspective schematic diagram of the present invention from another angle;

FIG. 3 to FIG. 5 are schematic diagrams of the implementation of the present invention;

FIG. 6 to FIG. 8 are schematic diagrams showing the implementation of the present invention manufactured using 3D printing technology;

FIG. 9 is a block flow diagram showing the manufacturing process of the present invention using 3D printing technology.

### DETAILED DESCRIPTION OF THE INVENTION

To further understand the features, technical means, and specific objectives and functions achieved by the present invention, a more detailed description is provided below, in conjunction with the accompanying drawings and specific implementations.

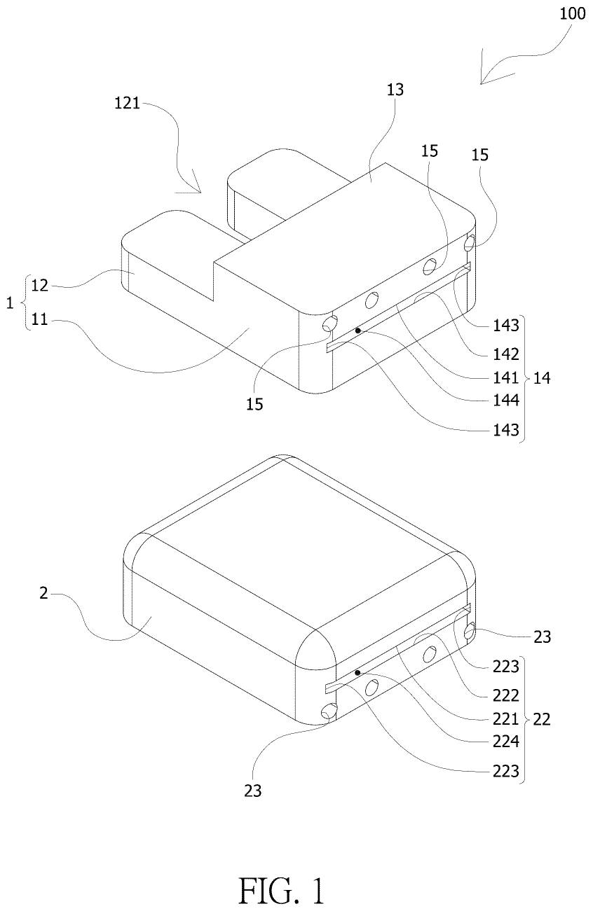

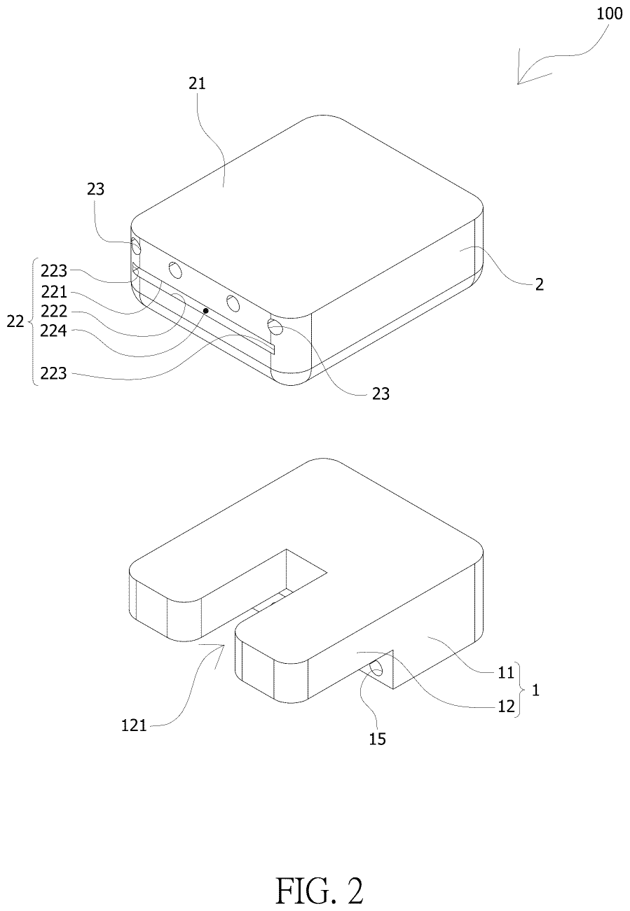

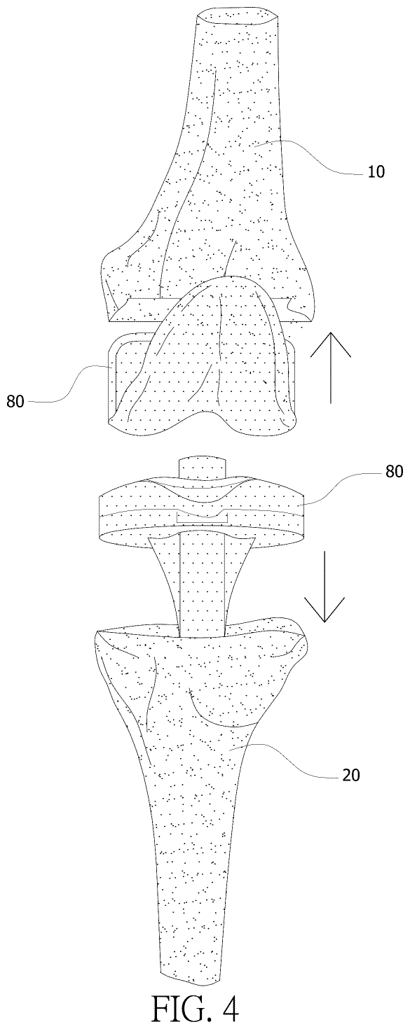

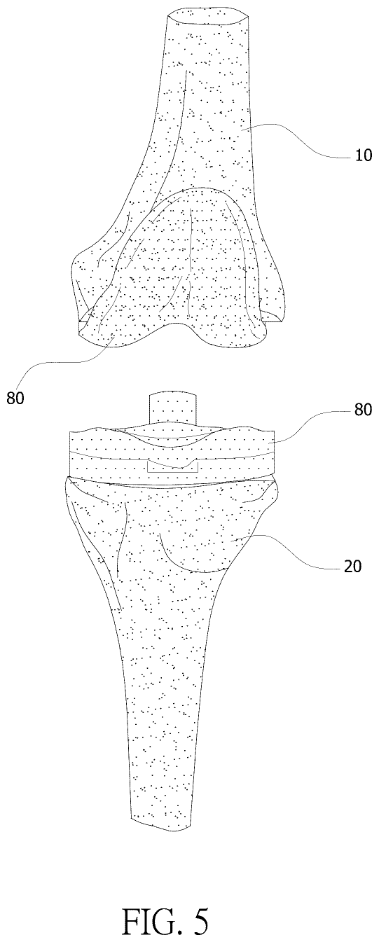

As shown in FIGS. 1 to 5, the present invention discloses a medical auxiliary device for joint bone replacement surgery, applied to the femur (10) and tibia (20). The medical auxiliary device (100) includes: a first auxiliary device (1), which has a vertical plate (11) that conforms to the femur (10) to be cut and a horizontal plate (12) extending laterally from the bottom of the vertical plate (11), also conforming to the femur (10). The top of the vertical plate (11) and horizontal plate (12) features a first fitting surface (13) that matches the shape of the femur (10), and one side of the vertical plate (11) is equipped with a first cutting groove (14) and multiple locking holes (15). The first cutting groove (14) allows an orthopedic saw (30) to cut the femur (10), and the locking holes (15) allow screws to pass through and fix the first auxiliary device (1) to the femur (10). Additionally, a second auxiliary device (2) corresponds to the first auxiliary device (1) and is fixed to the tibia (20). The bottom of the second auxiliary device (2) has a second fitting surface (21) matching the shape of the tibia (20), and one side of the second auxiliary device (2) is equipped with a second cutting groove (22) and multiple fixation holes (23). The second cutting groove (22) allows the orthopedic saw (30) to cut the tibia (20), and the fixation holes (23) allow screws to pass through and fix the second auxiliary device (2) to the tibia (20).

Through the application of the first auxiliary device (1) and the second auxiliary device (2), clinicians can perform osteotomy on the joint bones with greater precision, speed, and convenience. This makes the surgical procedure easier and more controllable, providing more precise and safer surgical outcomes.

Additionally, the first fitting surface (13) and second fitting surface (21) ensure that the first auxiliary device (1) and the second auxiliary device (2) tightly fit the joint bones. This allows for good contact between the medical auxiliary device and the bones, enabling clinicians to precisely remove damaged surfaces of the femur (10) and tibia (20), ensuring proper installation and stable support of the prosthesis (80) post-surgery, thereby restoring normal joint function and providing excellent surgical outcomes.

Furthermore, the first cutting groove (14), second cutting groove (22), locking holes (15), and fixation holes (23) accurately guide the orthopedic saw (30) and screws, providing stable fixation during surgery, ensuring surgical precision and safety. This allows clinicians to perform the surgery more easily, providing a controlled operating environment, improving surgical efficiency and success rates.

The horizontal plate (12) also includes a notch (121) through which cruciate ligaments can pass, allowing the ligaments to easily pass through without the need for additional tools or complicated procedures.

The vertical plate (11) and horizontal plate (12) are integrally formed, increasing the strength and stability of the medical auxiliary device (100). This reduces the number of connection points between components and minimizes potential failure points, improving overall durability and reliability, simplifying the manufacturing process, and reducing production costs.

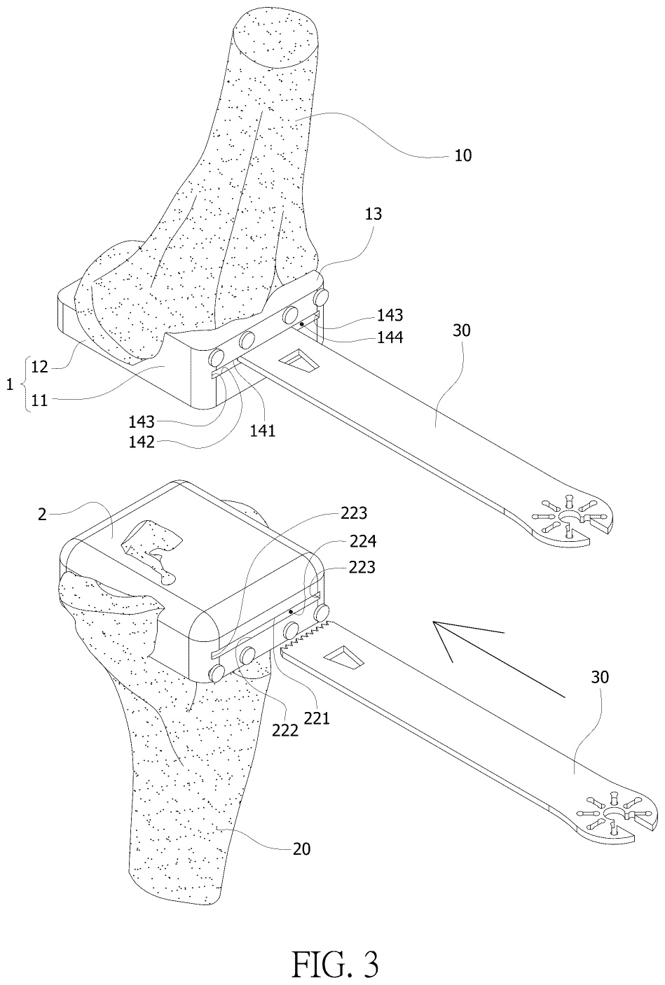

As further revealed in FIG. 3, the first cutting groove (14) consists of a first guide edge (141) positioned at the top of the first cutting groove (14), a second guide edge (142) positioned below the first guide edge (141), two slot walls (143) that connect perpendicularly to the guide edges and limit the operating direction and range of the orthopedic saw (30), and a saw blade opening (144) that penetrates the vertical plate (11) between the guide edges and slot walls. The second cutting groove (22) consists of a third guide edge (221) positioned at the top of the second cutting groove (22), a fourth guide edge (222) positioned below the third guide edge (221), two slot walls (223) that connect perpendicularly to the guide edges and limit the operating direction and range of the orthopedic saw (30), and a saw blade opening (224) that penetrates the second auxiliary device (2) between the guide edges and slot walls.

The combination of the first guide edge (141), second guide edge (142), third guide edge (221), fourth guide edge (222), first slot wall (143), and second slot wall (223) controls the operating direction of the orthopedic saw (30). The slot walls (143 and 223) limit the saw's lateral movement, including the direction and range of operation, to ensure accuracy. Additionally, the depth of operation can be controlled by limiting the saw's handle when it contacts the guide edges (141, 142, 221, 222), preventing damage to ligaments or nerves during surgery.

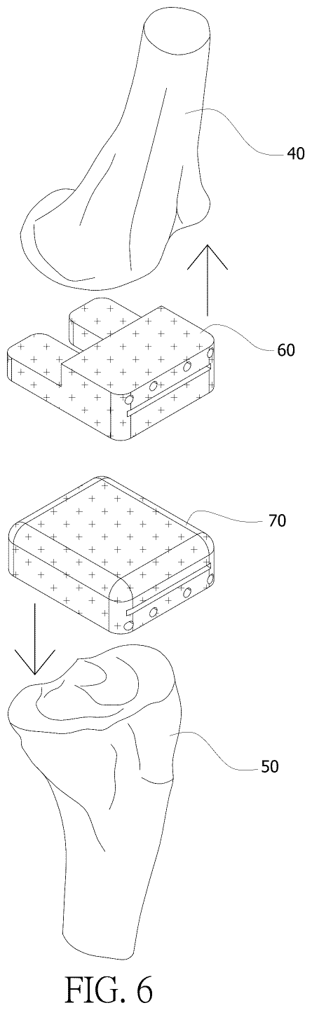

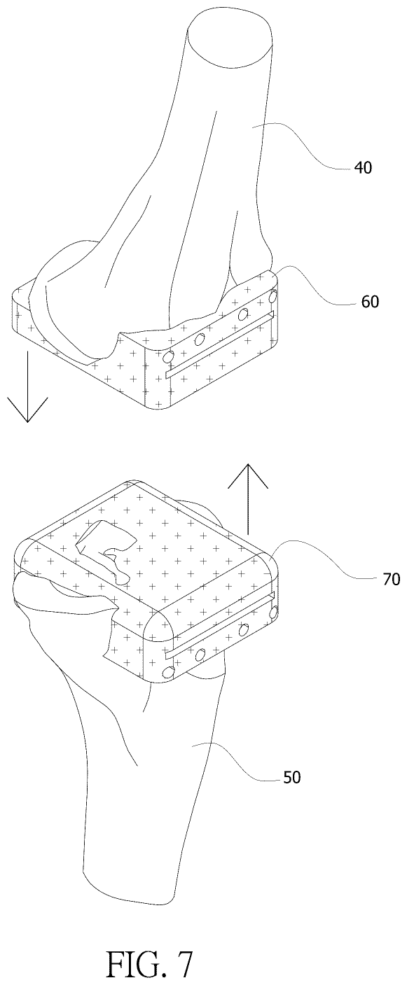

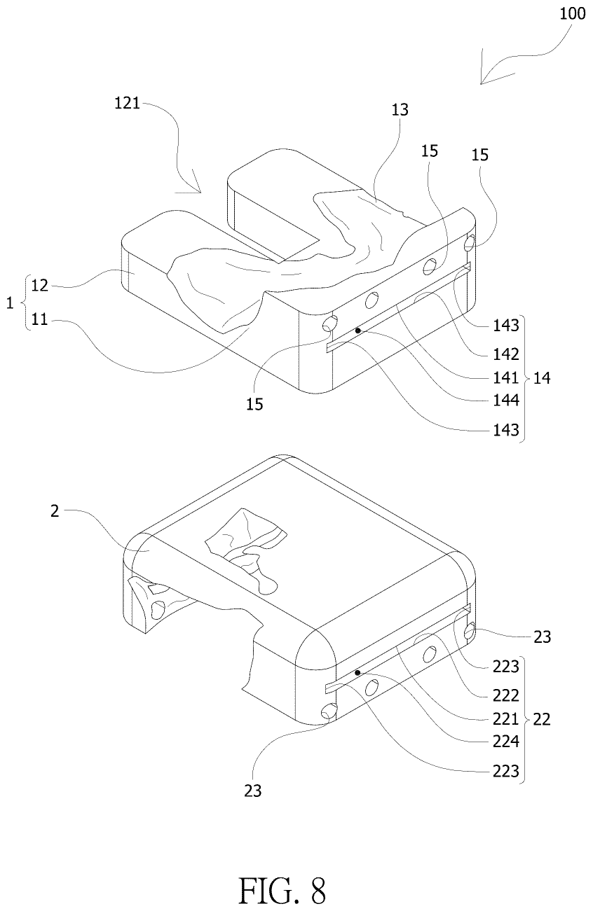

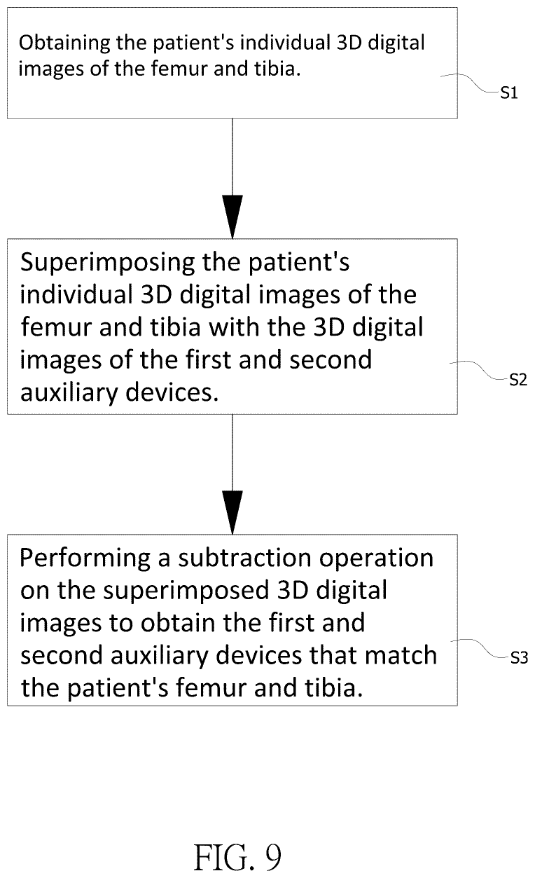

As shown in FIGS. 6 to 9, the medical auxiliary device for joint bone replacement surgery (100) is manufactured using 3D printing technology and involves the following steps:
Step 1 (S1): Obtain the patient's individual 3D digital images of the femur (40) and tibia (50);
Step 2 (S2): Superimpose the patient's femoral 3D digital image (40), tibial 3D digital image (50), and the 3D digital images of the first (60) and second auxiliary devices (70);
Step 3 (S3): Subtract the superimposed 3D digital images to obtain the first auxiliary device (1) and the second auxiliary device (2) that match the patient's femur (10) and tibia (20).

By customizing the device based on the patient's bone structure, a perfect match is ensured, providing better surgical outcomes.

From the above description, it is clear that the present invention provides a medical auxiliary device for joint bone replacement surgery, enabling clinicians to perform osteotomies with greater precision, speed, and convenience. The device makes the surgical procedure easier and more controllable, ensuring safer and more precise surgical outcomes, thus having significant value and importance for promoting the current trends in joint replacement surgery.

The above provides a detailed explanation of the methods, functions, and effects of the present invention. However, the described embodiments are only preferred examples and are not intended to limit the scope. Any modifications in line with the spirit of the invention should fall within the patent scope of this invention.

We claim:

1. A medical auxiliary device for joint bone replacement surgery, applied to a femur (10) and a tibia (20), the medical auxiliary device (100) comprising:
a first auxiliary device (1) having a vertical plate (11) that conforms to the femur (10) to be cut, and a horizontal plate (12) extending laterally from a bottom of the vertical plate (11) that conforms to the femur (10);
the vertical plate (11) and horizontal plate (12) having a first fitting surface (13) at a top that matches the shape of the femur (10); and
one side of the vertical plate (11) being provided with a first cutting groove (14) and a plurality of locking holes (15), the first cutting groove (14) allowing an orthopedic saw (30) to cut the femur (10), and the plurality of locking holes (15) allowing screws to pass through and fix the first auxiliary device (1) to the femur (10);
and a second auxiliary device (2) corresponding to the first auxiliary device (1) and fixed to the tibia (20), the second auxiliary device (2) having a second fitting surface (21) at the bottom that matches the shape of the tibia (20), and
one side of the second auxiliary device (2) being provided with a second cutting groove (22) and a plurality of fixation holes (23), the second cutting groove (22) allowing the orthopedic saw (30) to cut the tibia (20), and the plurality of fixation holes (23) allowing screws to pass through and fix the second auxiliary device (2) to the tibia (20).

2. The medical auxiliary device for joint bone replacement surgery as claimed in claim 1, wherein the first cutting groove (14) comprises a first guide edge (141) positioned at a top of the first cutting groove (14), a second guide edge (142) positioned below the first guide edge (141), two slot walls (143) positioned perpendicularly connecting the first guide edge (141) and the second guide edge (142) for restricting the operating direction and/or range of the orthopedic saw (30), and a first saw blade opening (144) penetrating the vertical plate (11) between the first guide edge (141), the second guide edge (142), and the first slot wall (143).

3. The medical auxiliary device for joint bone replacement surgery as claimed in claim 2, wherein the second cutting groove (22) comprises a third guide edge (221) positioned at the top of the second cutting groove (22), a fourth guide edge (222) positioned below the third guide edge (221), two slot walls (223) positioned perpendicularly connecting the third guide edge (221) and the fourth guide edge (222) for restricting the operating direction and/or range of the orthopedic saw (30), and a second saw blade opening (224) penetrating the second auxiliary device (2) between the third guide edge (221), the fourth guide edge (222), and the second slot wall (223).

4. The medical auxiliary device for joint bone replacement surgery as claimed in claim 3, wherein the horizontal plate (12) further comprises a notch (121) for the cruciate ligament to pass through.

5. The medical auxiliary device for joint bone replacement surgery as claimed in claim 4, wherein the vertical plate (11) and the horizontal plate (12) are integrally formed.

6. The medical auxiliary device for joint bone replacement surgery as claimed in claim 1, manufactured using 3D printing technology, comprising the following steps:
obtaining the patient's individual 3D digital images of the femur (40) and tibia (50);
superimposing the patient's femoral 3D digital image (40), tibial 3D digital image (50) with the 3D digital images of the first auxiliary device (60) and second auxiliary device (70);
performing a subtraction operation on the superimposed 3D digital images to obtain the first auxiliary device (1) and second auxiliary device (2) that match the patient's femur (10) and tibia (20).
